(19) United States
(12) Patent Application Publication
(10) Pub. No.: US 2024/0366359 A1
(43) Pub. Date: Nov. 7, 2024

(54) TRANSLUMINAL STENTS AND RELATED METHODS

(71) Applicant: Merit Medical Systems, Inc., South Jordan, UT (US)

(72) Inventors: Zeke Eller, Plano, TX (US); Evi Shiakolas, Dallas, TX (US)

(21) Appl. No.: 18/654,653

(22) Filed: May 3, 2024

Related U.S. Application Data
(60) Provisional application No. 63/500,544, filed on May 5, 2023.

(51) Int. Cl. A61F 2/04

(52) U.S. Cl. CPC A61F 2/04; A61F 2002/041; A61F 2002/044; A61F 2002/045; A61F 2230/0006; A61F 2250/0039

(57) ABSTRACT

Transluminal stents are disclosed herein. In some embodiments stents within the scope of this disclosure may comprise a first flared end, second flared end, and a middle region disposed between the first and second flared ends. The middle region may comprise a concave, curvilinear shape. The middle region may include a first portion, a second portion, and a circumferential ridge disposed between the first and second portions. The first flared end may include a first inner shoulder extending between the middle region and a first crest, a first opening, and a first outer taper extending between the first crest and the first opening. Methods of using the transluminal stents are also disclosed herein.

### RELATED APPLICATIONS

This application claims priority to U.S. Provisional Patent Application No. 63/500,544, filed May 5, 2023, and titled TRANSLUMINAL STENTS AND RELATED METHODS, which is incorporated herein by reference in its entirety.

TECHNICAL FIELD

This application generally relates to medical devices. More particularly, this application relates to non-vascular stents and related methods. More particularly, this application relates to transluminal stents and related methods of use.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 is a side view of an embodiment of a transluminal stent in an expanded state, such as when in use in vivo.

FIG. 2 is a side of the transluminal stent of FIG. 1 in crimped and elongated state, such as when loaded in a stent pod of a delivery catheter prior to deployment in vivo.

FIG. 3 is a side view of the shape of the transluminal stent of FIG. 1.

FIG. 4 is a side view of the transluminal stent of FIG. 1 in vivo.

FIG. 5 is a side view of the shape of another embodiment of a transluminal stent.

FIG. 6 is a side view of a portion of the shape of the transluminal stent of FIG. 5.

FIG. 7 is a side view of the shape of another embodiment of a transluminal stent.

### DETAILED DESCRIPTION

Transluminal and intraluminal stents are disclosed herein. In some embodiments, the stents described herein comprise a hollow body having an interior space and an exterior form or contour and comprising a concave, curvilinear middle region or portion that extends to a first flared end and also extends to an opposing second flared end. In some embodiments, the hollow body may have an hour glass shape. The interior space refers to the three dimensional space within the stent, while the exterior form or contour refers to the spatial contour of an exterior of the stent.

In some embodiments, the first flared end may comprise, a first crest, a first outer taper, and a first opening. A diameter of the first crest may be greater than a diameter of the middle region. The first outer taper may extend from the first crest to the first opening. The first opening may provide a first boundary between the interior space and the exterior form. The second flared end may be a mirror image of the first flared end reflected about a transverse plane that bisects the center of the middle region. In other words, the second flared end may comprise a second crest, a second outer taper, and a second opening. In other embodiments, the first flared end may further include a first inner shoulder.

In some embodiments, the hollow body may be characterized by a longitudinal plane that bisects the hollow body along its longitudinal axis, where the longitudinal plane encompasses a first circle defined, in part, by the middle region, a second circle defined, in part, by the first crest, and a third circle defined, in part, by the first outer taper. In such embodiments, a profile of at least a portion of the middle region may circumscribe a portion of a first arc of the first circle, a profile of at least a portion of the first crest may circumscribe a portion of a second arc of the second circle, and a profile of at least a portion of the first outer taper may circumscribe a portion of a third arc of the third circle. In some embodiments, the longitudinal plane encompasses a fourth circle defined by the first inner shoulder. A profile of at least a portion of the first inner shoulder may circumscribe a fourth arc of the fourth circle that lies in the longitudinal plane. In some embodiments, the longitudinal plane encompasses a fifth circle defined by a portion of the first inner shoulder. A profile of the portion of the first inner shoulder may circumscribe a fifth arc of the fifth circle that lies in the longitudinal plane.

In some embodiments, the hollow body may comprise braided or woven wires. In other embodiments, the hollow body may comprise a structure laser cut from a metal tube. The stents may be delivered via a delivery catheter. In certain embodiments, the stents may be self-expanding to self-transition from a crimped state to an expanded state when deployed from the delivery catheter. In other embodiments, the stents can be expanded by a balloon.

In certain circumstances, the transluminal stent may be used for draining one lumen of a patient into another lumen of a patient, such as, for example, transgastric or transduodenal drainage of a pancreatic cyst or pseudocyst, of a biliary tract, of a gallbladder. In other embodiments, the transluminal stent may be used intraluminally to transverse a stenosis or stricture of the patient's bowel. The hollow body can be disposed through a port in the stomach wall and a port in a wall of the pancreatic cyst such that the walls circumferentially surround the middle region. The first or distal flared end may be disposed in the pancreatic cyst and the second or proximal flared end may be disposed in the stomach. The hourglass shape of the hollow body can enhance the appositional relationship of the walls by directing the walls toward one another and preventing separation of the walls. Fluid and/or necrotic tissue within the pancreatic cyst or pseudocyst can flow through the hollow body into the stomach. In other circumstances, the stents may be utilized to facilitate passage of scopes or other medical devices from the stomach into the pancreas or other anotomical areas. In still other circumstances, the stents may be utilized in an intraluminal function to provide a passage through a stenosis of a patient's intestine. Thus it will be understood that the stent may be used in a variety of ways, including, but not limited to, biliary drainage, pancreatic fluid collection drainage, gallbladder drainage, gastro-gastrostomy, gastro-jejunostomy, pancreatic duct drainage, intra-abdominal abscess drainage, natural orifice transluminal endoscopic surgery (NOTES), and for gastrointestinal strictures. It is also contemplated that the stent can be disposed in the colon, pylorus, duodeum, or in other areas of a patient's body, such as the esophagus as an esophageal stent.

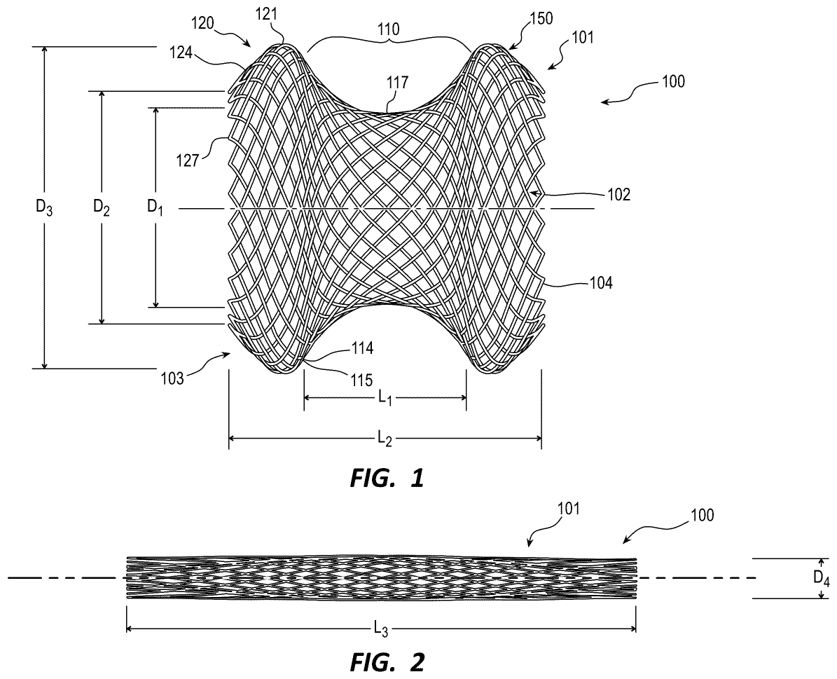

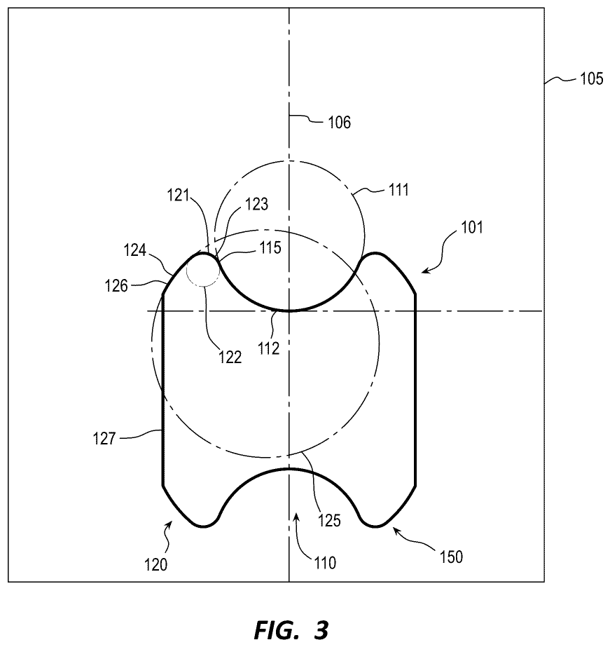

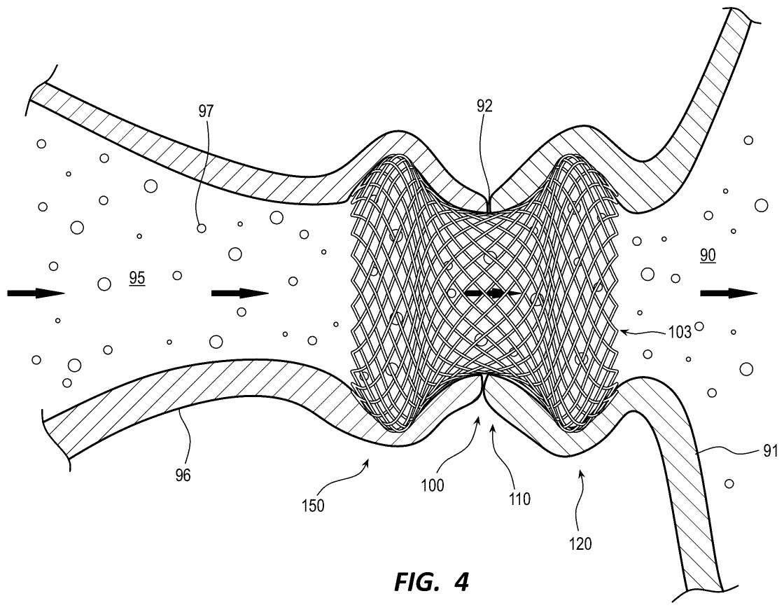

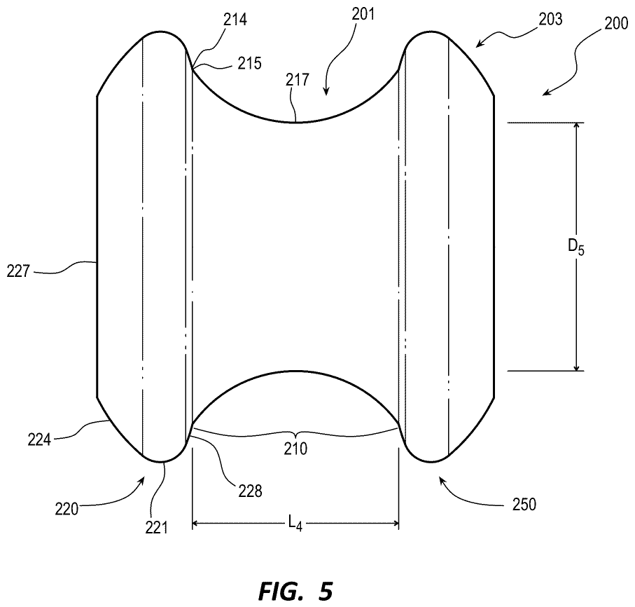

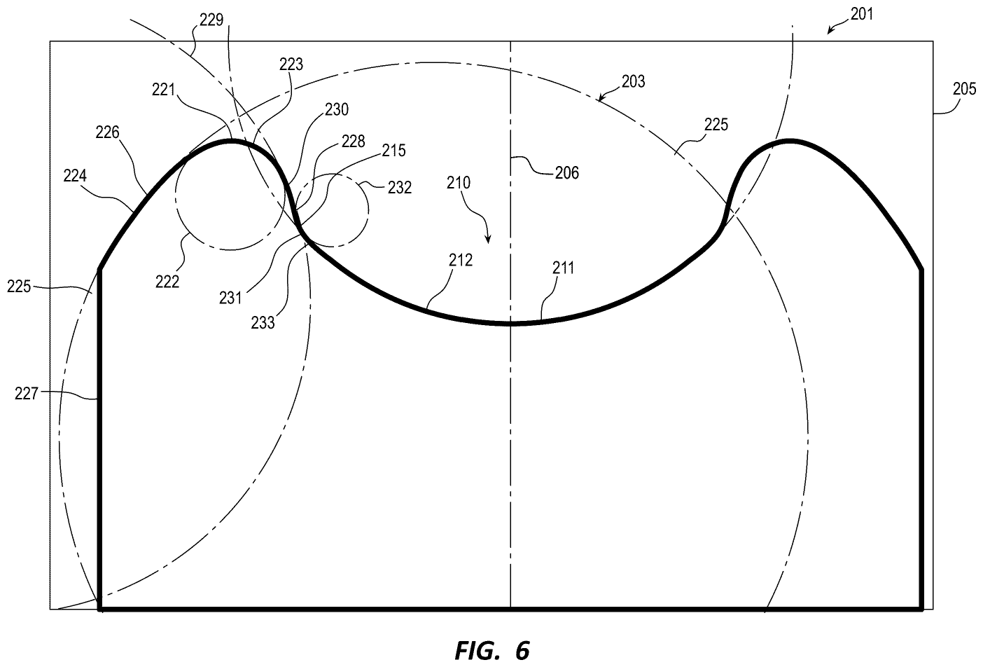

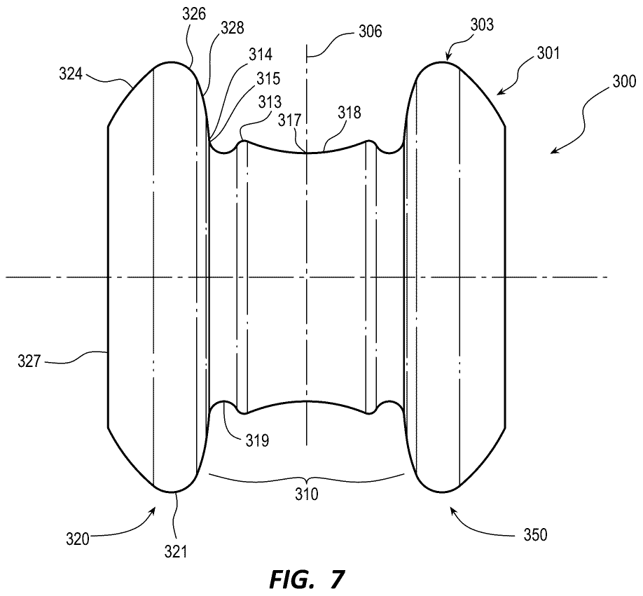

FIG. 1 illustrates an embodiment of a transluminal stent in an expanded state, such as when in use in vivo. FIG. 2 illustrates the transluminal stent of FIG. 1 in a crimped and elongated state, such as when loaded in a stent pod of a delivery catheter prior to deployment in vivo. FIG. 3 illustrates the shape of the transluminal stent of FIG. 1. FIG. 4 illustrates the transluminal stent of FIG. 1 in vivo. FIG. 5 illustrates the shape of another embodiment of a transluminal stent. FIG. 6 illustrates a portion of the shape of the transluminal stent of FIG. 5. FIG. 7 illustrates the shape of another embodiment of a transluminal stent. It will be readily understood that the components of the embodiments as generally described and illustrated in the figures herein could be arranged and designed in a wide variety of different configurations. Thus, the following more detailed description of various embodiments, as represented in the figures, is not intended to limit the scope of the present disclosure, but is merely representative of various embodiments. While various aspects of the embodiments are presented in drawings, the drawings are not necessarily drawn to scale unless specifically indicated.

FIG. 1 illustrates an embodiment of a transluminal stent 100 in an expanded state, such as when in use in vivo. In one embodiment, the transluminal stent 100 may be a self-expanding stent that expands from a crimped state (shown in FIG. 2) to the expanded state when deployed from a delivery catheter. In another embodiment, the transluminal stent 100 may be a balloon expandable stent that is expanded by a balloon of a delivery catheter. The transluminal stent 100 comprises a hollow body 101 having an interior space 102 (see FIG. 3) and an exterior form or contour 103. The hollow body 101 comprises a middle region or portion 110 that extends to a first flared end 120 and also extends to an opposing second flared end 150 that is a mirror image of the first flared end 120. In other embodiments, the second flared end 150 may comprise a form or contour different than the first flared end 120. For example, dimensions, such as diameters and/or lengths, of the second flared end 150 may be larger or smaller than similar dimensions of the first flared end 120. The first flared end 120 may be disposed and retained within one region of a patient's body (e.g., a pancreatic cyst) in the expanded state, the second flared end 150 may be disposed and retained within another region of the patient's body (e.g., the stomach) in the expanded state. In one example, the walls of the pancreatic cyst and the stomach can be appositional at the middle region 110. Fluid and other matter may flow through the interior space 102 from the pancreatic cyst to the stomach.

In some embodiments, the overall length L₂ of the transluminal stent 100 in the expanded state may range from about 8 millimeters to about 36 millimeters, including ranging from about 12 millimeters to about 24 millimeters, about 14 millimeters to about 22 millimeters, about 20 millimeters to about 34 millimeters, about 20 millimeters to about 30 millimeters, or about 21 millimeters to about 29 millimeters. In certain embodiments, the hollow body 101 comprises a braided or woven structure of one or more wires 104. The wires 104 may be woven or braided in any suitable pattern to provide a self-expanding stent that resists radial compression. The braid pattern may be a one-wire, two-over, two-under braid pattern (referred to as a “one over two” pattern), which means that a single strand passes over two strands (or two different portions of itself, such as in a single wire braid design) and then under two other strands (or yet two other portions of itself, such as in a single wire braid design). Alternative braid patterns may be used as well, such as a one-wire, one-over, one-under braid pattern (referred to as a “one over one” pattern). Other possible braid patterns include the diamond two-wire, one-over, one-under braid pattern, the hook and cross braid pattern, and the diamond two, two-over, two-under braid pattern.

In some embodiments, the braided or woven wires 104 may be braided or woven in a given pattern in accordance with an appropriate braid design, such as a closed-loop braid design, a single wire woven design, an endless braid design, or the like. The braided or woven wires 104 forming the transluminal stent 100 may comprise any suitable material known in the art, including plastics and memory alloys. In some embodiments, the braided or woven wires 104 may be a nickel-titanium alloy, such as Nitinol, including ASTM F2063. In one embodiment, the thickness of a memory alloy strand of the braided or woven wires 104 may be about 0.07 millimeter to about 0.23 millimeter. Generally speaking, although not required, smaller wires may be used with smaller diameter stents and larger diameter wires may be used with larger diameter stents.

As illustrated, the middle region 110 comprises a concave, curvilinear shape that extends from a first end 114 to a midpoint 117. The concave, curvilinear shape can be configured to enhance the apposition of tissues surrounding the middle region 110 in vivo. The first end 114 includes a first inflection point 115 where the concave, curvilinear shape transitions to a convex, curvilinear shape of a first crest 121. In certain embodiments, the diameter D₁ of the middle region 110 at the midpoint 117 in the expanded state may range from about 6 millimeters to about 26 millimeters, including ranging from about 6 millimeters to about 24 millimeters, about 10 millimeters to about 22 millimeters, or about 10 millimeters to about 20 millimeters. In some embodiments, the length L₁ of the middle region 110 in the expanded state may range from about 6 millimeters to about 20 millimeters, including ranging from about 8 millimeters to about 16 millimeters, or about 10 millimeters to about 16 millimeters.

The first flared end 120 comprises the first crest 121, a first outer taper 124, and a first opening 127. The first crest 121 extends from the first end 114 of the middle region 110 or first inflection point 115 to the first outer taper 124. The first outer taper 124 extends between the first crest 121 and the first opening 127. The first opening 127 provides a first boundary between the interior space 102 and the exterior form 103. The diameter D₃ of the first crest 121 in the expanded state may range from about 10 millimeters to about 45 millimeters, including ranging from about 10 millimeters to about 38 millimeters, about 17 millimeters to about 36 millimeters, or about 17 millimeters to about 29 millimeters. For example, in some embodiments, when D₁ is 6 millimeters, D₃ is about 10.4 millimeters; when D₁ is 8 millimeters, D₃ is about 13.8 millimeters; when D₁ is 10 millimeters, D₃ is about 17 millimeters; when D₁ is 15 millimeters, D₃ is about 26 millimeters; when D₁ is 16 millimeters, D₃ is about 25.2 millimeters or 27.2 millimeters; when D₁ is 20 millimeters, D₃ is about 32 millimeters; when D₁ is 22 millimeters, D₃ is about 32 millimeters or about 36 millimeters; and when D₁ is 24 millimeters, D₃ is about 33 millimeters or about 34 millimeters. In the illustrated embodiment, the diameter D₃ of the first crest 121 is greater than the diameter D₁ of the middle region 110 by 1.7 times or less.

In the illustrated embodiment, a diameter D₂ of the first opening 127 is greater than the diameter D₁ of the middle region 110, but less than the diameter D₃ of the first crest 121. This can facilitate removal of the transluminal stent 100 from a mandrel during manufacture. This can also reduce particle entrapment during drainage via the interior space 102. Particles and fluid can tend to fill the interior space 102 of the first and second flared ends 120 and 150 during use, such as drainage of the gall bladder, a biliary tract, or a pancreatic cyst. When the diameter D₂ of the first opening 127 is the same diameter or smaller than the diameter D₁ of the middle region 110, this can make removal of fluid and particles from the interior space 102 of the first flared end 120 difficult. Increasing the diameter D₂ of the first opening 127, relative to the diameter D₁ of the middle region 110, can facilitate particle and fluid removal from the interior space 102 of the first flared end 120. Because the second flared end 150 is a mirror image of the first flared end 120, the described advantages of the configuration of the first flared end 120 apply to the second flared end 150. In certain embodiments, the diameter D₂ of the first opening 127 in the expanded state may range from about 7 millimeters to about 31 millimeters, including ranging from about 7 millimeters to about 26 millimeters, about 11 millimeters to about 25 millimeters, about 11 millimeters to about 21 millimeters. In some embodiments, the diameter D₂ of the first opening 127 is less than the diameter D₃ of the first crest 121 by about 0.7 times.

FIG. 2 illustrates the transluminal stent 100 in an elongated and crimped state, such as when loaded in a stent pod of a delivery catheter prior to deployment in vivo. The length L₃ of the transluminal stent 100 in the elongated and crimped state may range from about 34 millimeters to about 75 millimeters, including ranging from about 40 millimeters to about 74 millimeters, about 46 millimeters to about 72 millimeters, about 47 millimeters to about 70 millimeters, or from about 60 millimeters to 70 millimeters. In some embodiments, the diameter D₄ of the transluminal stent 100 in the elongated and crimped state may be from about 3 millimeters to about 4 millimeters, such as less than about 3.7 millimeters, or about 3.5 millimeters. In some embodiments, the transluminal stent 100 may be elongated and radially crimped to fit within a stent pod of a 10.5 French delivery catheter. In other embodiments, the transluminal stent 100 may be elongated and radially crimped to be delivered from a delivery catheter having a size as small as 9 French.

FIG. 3 illustrates a contour of the hollow body 101 bisected in a longitudinal plane 105 along its longitudinal axis. The hollow body 101 is characterized by the longitudinal plane 105 that encompasses a first circle 111 defined by the middle region 110. In the illustrated embodiment, a profile of at least a portion of the middle region 110 circumscribes a first arc 112 of the first circle 111. The diameter of the first circle 111 for the transluminal stent 100, where D₁ is 15 millimeters and L₁ is 10 millimeters, may be about 13.5 millimeters to about 15.1 millimeters (e.g., such as about 14.3 millimeters or about 14.4 millimeters) and an arc length of the first arc 112 may be about 15.7 millimeters to about 17.3 millimeters (e.g., such as about 16.5 millimeters).

The longitudinal plane 105 encompasses a second circle 122 defined by the first crest 121. In the illustrated embodiment, a profile of at least a portion of the first crest 121 circumscribes a second arc 123 of the second circle 122 that lies in the longitudinal plane 105. The diameter of the second circle 122 for the transluminal stent 100, where D₁ is 15 millimeters and L₁ is 10 millimeters, may be about 3.1 millimeters to about 3.5 millimeters (e.g., such as about 3.3) and arc length of the second arc 123 may be about 2.8 millimeters to about 3.2 millimeters (e.g., such as about 3.0 millimeters).

The longitudinal plane 105 encompasses a third circle 125 defined by the first outer taper 124. In the illustrated embodiment, a profile of at least a portion of the first outer taper 124 circumscribes a third arc 126 of the third circle 125 that lies in the longitudinal plane 105. The diameter of the third circle 125 for the transluminal stent 100, where D₁ is 15 millimeters and L₁ is 10 millimeter, may be about 20.7 millimeters to about 22.7 millimeters (e.g., such as about 21.7 millimeters) and an arc length of the third arc 126 may be about 4.2 millimeters to about 4.8 millimeters (e.g., such as about 4.5 millimeters).

As illustrated in FIG. 3, the first circle 111 intersects the third circle 125 and tangentially intersects the second circle 122. The second circle 122 is disposed within the third circle 125 such that the second circle 122 tangentially intersects the first circle 111 at the inflection point 115. The first arc 112 or middle region 110 extends from the midpoint 117 to the inflection point 115. The second arc 123 or first crest 121 extends from the inflection point 115 to the third arc 126 or from the inflection point 115 to the first outer taper 124. The third arc 126 or first outer taper 124 extends from the second arc 123 to the first opening 127 or from the first crest 121 to the first opening 127.

In the illustrated embodiments, the profile of the second flared end 150 is a mirror image of the first flared end 120 where the second flared end 150 is reflected about a transverse plane 106 bisecting the middle region 110. In alternative embodiments, the shape of the second flared end 150 may differ from the shape of the first flared end 120, in some cases, significantly.

The stents disclosed herein, such as the transluminal stent 100, may be used for draining one lumen of a patient into another lumen of a patient, such as, for example, transgastric or transduodenal drainage of a pancreatic cyst or pseudocyst, of a biliary tract, of a gallbladder. In other embodiments, the transluminal stent 100 may be used intraluminally such as to transverse a stenosis or stricture of the patient's bowel. As illustrated in FIG. 4, an access port 92 may be created between a first lumen 90 of the patient and a second lumen 95 of the patient. For example, natural orifice transluminal endoscopic surgery (NOTES) may be used to create the access port 92 between the lumens. The first lumen 90 may be the gastrointestinal tract (for example, the esophagus, stomach, pylorus, or bowel) of the patient. The second lumen 95 may be the gallbladder, a pancreatic cyst or pseudocyst, a biliary tract, or some other lumen that needs drainage or to be accessed. A delivery catheter (not shown) with the transluminal stent 100 loaded in the elongated and crimped state, as shown in FIG. 2, may be introduced through the working channel of an endoscope or other device (not shown) and through the access port 92. The second flared end 150 (in its crimped and elongated state) may be introduced through the access port 92 into the second lumen 95 and then the second flared end 150 deployed from the delivery catheter so that the second flared end 150 transitions to the expanded state, as shown in FIG. 1, and is secured against a luminal wall 96 of the second lumen 95. The middle region 110 and the first flared end 120 can then be deployed from the delivery catheter so that the middle region 110 and the first flared end 120 transition to the expanded state, as shown in FIG. 1. The first flared end 120 is secured against a luminal wall 91 of the first lumen 90 and the tissues of the access port 92 circumferentially surround the middle region 110. The concave, curvilinear shape of the middle region 110 can facilitate apposition and prevent movement of the luminal walls 91, 96. In its expanded state, the interior space 102 of the transluminal stent 100 provides fluidic communication with the first and second lumens 90, 95. The second lumen 95 can passively drain fluid or material 97 into the first lumen 90 or the second lumen 95 can be actively drained by insertion of other tools through the interior space 102 into the second lumen 95 to remove the fluid or material 97 from the second lumen 95 (such as, for example, gallstones or malignant or necrotic tissue).

FIGS. 5 and 6 depict a shape or contour of an embodiment of a transluminal stent 200 that resembles the transluminal stent 100 described above in certain respects. Accordingly, like features are designated with like reference numerals, with the leading digit incremented to “2.” For example, the embodiment depicted in FIGS. 5 and 6 includes a middle region 210 that may, in some respects, resemble the middle region 110 of FIG. 1. Relevant disclosure set forth above regarding similarly identified features thus may not be repeated hereafter. Moreover, specific features of the transluminal stent 100 and related components shown in FIGS. 1-4 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the transluminal stent 200 and related components depicted in FIGS. 5 and 6. Any suitable combination of the features, and variations of the same, described with respect to the transluminal stent 100 and related components illustrated in FIGS. 1-4 can be employed with the transluminal stent 200 and related components of FIGS. 5 and 6, and vice versa. This pattern of disclosure applies equally to further embodiments depicted in subsequent figures and described hereafter, wherein the leading digits may be further incremented.

As shown in FIG. 5, the shape or contour of a hollow body 201 of the transluminal stent 200 includes an exterior form or contour 203. The contour 203 comprises a middle region or portion 210 that extends to a first flared end 220 and also extends to an opposing second flared end 250 that is a mirror image of the first flared end 220.

As illustrated, the middle region 210 comprises a concave, curvilinear shape that extends from a first end 214 to a midpoint 217. The first end 214 includes a first inflection point 215 where the concave, curvilinear shape transitions to a convex, curvilinear shape of a first inner shoulder 228. In certain embodiments, the diameter D₅ of the middle region 210 at the midpoint 217 in the expanded state may range from about 6 millimeters to about 24 millimeters, including ranging from about 10 millimeters to about 20 millimeters. In some embodiments, the length L₄ of the middle region 210 in the expanded state may range from about 6 millimeters to about 20 millimeters, including ranging from about 8 millimeters to about 16 millimeters or about 10 millimeters to about 16 millimeters.

The first flared end 220 comprises a first crest 221, a first outer taper 224, a first opening 227, and the first inner shoulder 228. The first inner shoulder 228 extends from the first end 214 of the middle region 210 or first inflection point 215 to the first crest 221. The first crest 221 extends between the first inner shoulder 228 and the first outer taper 224. The first outer taper 224 extends between the first crest 221 and the first opening 227.

FIG. 6 illustrates a portion of the contour 203 of the hollow body 201 bisected in a longitudinal plane 205 along its longitudinal axis. The hollow body 201 is characterized by the longitudinal plane 205 that encompasses a first circle 211 defined by the middle region 210. In the illustrated embodiment, a profile of at least a portion of the middle region 210 circumscribes a first arc 212 of the first circle 211. The diameter of the first circle 211 for the transluminal stent 200, where D₅ is 15 millimeters and L₄ is 10 millimeters may be about 16 millimeters to about 17.6 millimeters (e.g., such as about 16.8 millimeters) and an arc length of the first arc 212 may be about 12.6 millimeters to about 14 millimeters (e.g., such as about 13.3 millimeters).

The longitudinal plane 205 encompasses a second circle 222 defined by the first crest 221. In the illustrated embodiment, a profile of at least a portion of the first crest 221 circumscribes a second arc 223 of the second circle 222 that lies in the longitudinal plane 205. The diameter of the second circle 222 for the transluminal stent 200, where D₅ is 15 millimeters or 16 millimeters and L₄ is 10 millimeters may be about 3.1 millimeters to about 3.5 millimeters (e.g., such as about 3.3 millimeters) and an arc length of the second arc 223 may be about 2.9 millimeters to about 3.3 millimeters (e.g., such as about 3.1 millimeters).

The longitudinal plane 205 encompasses a third circle 225 defined by the first outer taper 224. In the illustrated embodiment, a profile of at least a portion of the first outer taper 224 circumscribes a third arc 226 of the third circle 225 that lies in the longitudinal plane 205. The diameter of the third circle 225 for the transluminal stent 200, where D₅ is 15 millimeters and L₄ is 10 millimeters may be about 22.6 millimeters to about 24.6 millimeters (e.g., such as about 23.6 millimeters) and an arc length of the third arc 226 may about 4.2 millimeters to about 4.8 millimeters (e.g., such as about 4.5 millimeters). The diameter of the third circle 225 for the transluminal stent 200, where D₅ is 16 millimeters and L₄ is 10 millimeters may be about 20.7 millimeters to about 22.7 millimeters (e.g., such as about 21.7 millimeters) and an arc length of the third arc 226 may about 4.2 millimeters to about 4.8 millimeters (e.g., such as about 4.5 millimeters).

The longitudinal plane 205 encompasses a fourth circle 229 defined by the first inner shoulder 228. In the illustrated embodiment, a profile of at least a portion of the first inner shoulder 228 circumscribes a fourth arc 230 of the fourth circle 229 that lies in the longitudinal plane 205. The diameter of the fourth circle 229 for the transluminal stent 200, where D₅ is 15 millimeters and L₄ is 10 millimeters may be about 17.4 millimeters to about 19.4 millimeters (e.g., such as about 18.4 millimeters) and an arc length of the fourth arc 230 may be about 1.2 millimeter to about 1.4 millimeters (e.g., such as about 1.3 millimeters). The diameter of the fourth circle 229 for the transluminal stent 200, where D₅ is 16 millimeters and L₄ is 10 millimeters may be about 17.4 millimeters to about 19.4 millimeters (e.g., such as about 18.4 millimeters) and an arc length of the fourth arc 230 may be about 1.5 millimeter to about 2.3 millimeters (e.g., such as about 1.9 millimeters).

The longitudinal plane 205 encompasses a fifth circle 232 defined by a portion 231 of the first inner shoulder 228. In the illustrated embodiment, a profile of the portion 231 circumscribes a fifth arc 233 of the fifth circle 232 that lies in the longitudinal plane 205. The diameter of the fifth circle 232 for the transluminal stent 200, where D₅ is 15 millimeters and L₄ is 10 millimeters may be about 1.9 millimeters to about 2.1 millimeters (e.g., such as about 2 millimeters) and an arc length of the fifth arc 233 may be about 0.5 millimeters to about 0.6 millimeters (e.g., such as about 0.54 millimeters). The diameter of the fifth circle 232 for the transluminal stent 200, where D₅ is 16 millimeters and L₄ is 10 millimeters may be about 1.9 millimeters to about 2.1 millimeters (e.g., such as about 2 millimeters) and an arc length of the fifth arc 233 may be about 0.3 millimeters to about 0.5 millimeters (e.g., such as about 0.4 millimeters).

As illustrated in FIG. 6, the first circle 211 intersects the third circle 225 and tangentially intersects the second circle 222, fourth circle 229, and fifth circle 232. The fifth circle 232 is disposed within the first circle 211 and the third circle 225. The third circle 225 overlaps a portion of the fourth circle 229 and the second circle 222 is disposed within the overlapping portion of the third circle 225 and fourth circle 229 such that the second circle 222 tangentially intersects the third circle 225 and the fourth circle 229 at the inflection point 215. The first arc 212 or middle region 210 extends from the midpoint 217 to the fifth arc 233 or from the midpoint to the portion 231 of the first inner shoulder 228. The fourth arc 230 or first inner shoulder 228 extends from the fifth arc 233 to the second arc 223 or from the middle region 210 to the first crest 221. The second arc 223 or first crest 221 extends from the fourth arc 230 to the third arc 226 or from the first inner shoulder 228 to the first outer taper 224. The third arc 226 or first outer taper 224 extends from the second arc 223 to the first opening 227 or from the first crest 221 to the first opening 227.

In the illustrated embodiments, the profile of the second flared end 250 is a mirror image of the first flared end 220 where the second flared end 550 is reflected about a transverse plane 206 bisecting the middle region 210. In alternative embodiments, the shape of the second flared end 250 may differ from the shape of the first flared end 220, in some cases, significantly.

FIG. 7 illustrates a shape or contour of another embodiment of a transluminal stent 300. As shown in FIG. 7, the shape or contour of a hollow body 301 of the transluminal stent 300 includes an exterior form or contour 303. The contour 303 comprises a middle region or portion 310 that extends to a first flared end 320 and also extends to an opposing second flared end 350 that is a mirror image of the first flared end 320.

As illustrated, the middle region 310 comprises a first concave, curvilinear portion 318, a first circumferential ridge 313, and a second concave, curvilinear portion 319. The first curvilinear portion 318 extends from a midpoint 317 to the first circumferential ridge 313. The second curvilinear portion 319 extends from the first circumferential ridge 313 to the first end 314 or first inner shoulder 328. The first end 314 includes a first inflection point 315 where the second concave, curvilinear shape transitions to a convex, curvilinear shape of the first inner shoulder 328. In certain embodiments, the minimum diameter of the first curvilinear portion 318 at the midpoint 317 may be smaller than the diameter of the first circumferential ridge 313. The minimum diameter of the second curvilinear portion 319 may also be smaller than the diameter of the first circumferential ridge 313. In some embodiments, the minimum diameter of the first curvilinear portion 318 at the midpoint 317 is also less than the minimum diameter of the second curvilinear portion 319. In other embodiments, the minimum diameter of the first curvilinear portion 318 at the midpoint 317 is substantially the same as the minimum diameter of the second curvilinear portion 319.

The first flared end 320 comprises a first crest 321, a first outer taper 324, a first opening 327, and the first inner shoulder 328. The first inner shoulder 328 extends from the first end 314 of the middle region 310 or first inflection point 315 to the first crest 321. The first crest 321 extends between the first inner shoulder 328 and the first outer taper 324. The first outer taper extends between the first crest 321 and the first opening 327.

In the illustrated embodiment, the profile of the second flared end 350 and an opposing half of the middle region 310 is a mirror image of the first flared end 320, the first curvilinear portion 318, the first circumferential ridge 313, and the second curvilinear portion 319. In alternative embodiments, the shape of the second flared end 350 may differ from the shape of the first flared end 320, in some cases, significantly.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, only a portion of a method described herein may be a separate method. Stated otherwise, some methods may include only a portion of the steps described in a more detailed method.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

The phrase “in communication with” is used in its ordinary sense, and is broad enough to refer to any suitable communication or other form of interaction between two or more entities, including mechanical, electrical, magnetic, electromagnetic, fluid, and thermal interaction. Two components may interact with each other even though they are not in direct contact with each other. For example, two components may be in communication with each other through an intermediate component.

Recitation in the claims of the term “first” with respect to a feature or element does not necessarily imply the existence of a second or additional such feature or element. It will be apparent to those having skill in the art that changes may be made to the details of the above-described embodiments without departing from the underlying principles of this disclosure.

“Fluid” is used in its broadest sense, to refer to any fluid, including both liquids and gases as well as solutions, compounds, suspensions, etc., which generally behave as fluids.

References to approximations are made throughout this specification, such as by use of the term “about.” For each such reference, it is to be understood that, in some embodiments, the value, feature, or characteristic may be specified without approximation. For example, where the qualifier such as “about” is used, this term includes within its scope the qualified words in the absence of its qualifier. For example, where the term “about” is recited with respect to a feature, it is understood that in further embodiments, the feature can have a precise configuration.

The terms “a” and “an” can be described as one, but not limited to one. For example, although the disclosure may recite a housing having “a stopper,” the disclosure also contemplates that the housing can have two or more stoppers.

Unless otherwise stated, all ranges include both endpoints and all numbers between the endpoints.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A stent comprising:
a hollow body comprising:
an interior space;
an exterior contour;
a first flared end, a second flared end, and a middle region that extends between the first flared end and the second flared end,
wherein the middle region comprises a concave, curvilinear shape.

2. The stent of claim 1, wherein the first flared end comprises:
a first crest extending from the middle region;
a first opening; and
a first outer taper extending between the first crest and the first opening.

3. The stent of claim 2, wherein the first flared end further comprises a first inner shoulder extending between the middle region and the first crest.

4. The stent of claim 1, wherein the middle region comprises:
a first portion;
a second portion; and
a first circumferential ridge disposed between the first portion and the second portion.

5. The stent of claim 4, wherein the diameter of the first circumferential ridge is larger than a diameter of the first portion of the middle region.

6. The stent of claim 2, wherein the hollow body further comprises a longitudinal plane that bisects the hollow body along a longitudinal axis of the hollow body,
wherein the longitudinal plane comprises a first circle defined at least in part by the middle region, and
wherein a profile of at least a portion of the middle region circumscribes a first arc of the first circle.

7. The stent of claim 6, wherein the longitudinal plane further comprises a second circle defined at least in part by the first crest, a third circle defined at least in part by the first outer taper, and a fourth circle defined at least in part by a first inner shoulder.

8. The stent of claim 7, wherein a profile of at least a portion of the first crest circumscribes a second arc of the second circle, a profile of at least a portion of the first outer taper circumscribes a third arc of the third circle, and a profile of at least a portion of the first inner shoulder circumscribes a fourth arc of the fourth circle.

9. The stent of claim 7, wherein the longitudinal plane further comprises a fifth circle defined at least in part by the first inner shoulder, a profile of a portion of the first inner shoulder circumscribing a fifth arc of the fifth circle.

10. A braided stent, comprising:
a hollow body comprising:
an interior space;
an exterior contour;
a first flared end, a second flared end, and a middle region that extends between the first flared end and the second flared end,
wherein the middle region comprises a concave, curvilinear shape, and
wherein the first flared end comprises a first crest extending from the middle region, a first opening, and a first outer taper extending between the first crest and the first opening.

11. The braided stent of claim 10, wherein the middle region further comprises:
a first portion;
a second portion; and
a first circumferential ridge disposed between the first portion and the second portion.

12. The braided stent of claim 11, wherein the diameter of the first circumferential ridge is larger than a diameter of the first portion of the middle region.

13. The braided stent of claim 9, wherein the first flared end further comprises a first inner shoulder extending between the middle region and the first crest.

14. The braided stent of claim 10, wherein the hollow body further comprises a longitudinal plane that bisects the hollow body along a longitudinal axis of the hollow body, the longitudinal plane comprising:
a first circle defined at least in part by the middle region, a profile of at least a portion of the middle region circumscribing a first arc of the first circle;
a second circle defined at least in part by the first crest, a profile of at least a portion of the first crest circumscribing a second arc of the second circle; and
a third circle defined at least in part by the first outer taper, a profile of at least a portion of the first outer taper circumscribing a third arc of the third circle.

15. The braided stent of claim 14, wherein the longitudinal plane further comprises one or more circles defined at least in part by a first inner shoulder, one or more profiles of at least a portion of the first inner shoulder circumscribing one or more arcs of the one or more circles defined at least in part by the first inner shoulder.

16. A transluminal stent, comprising
a hollow body comprising:
an interior space;
an exterior contour;
a first flared end, a second flared end, and a middle region that extends between the first flared end and the second flared end,
wherein the middle region comprises:
a concave, curvilinear shaped first portion;
a concave, curvilinear shaped second portion; and
a first circumferential ridge disposed between the first portion and the second portion.

17. The transluminal stent of claim 16, wherein the first flared end comprises:
a first crest extending from the second portion;
a first opening; and
a first outer taper extending between the first crest and the first opening.

18. The transluminal stent of claim 17, wherein the first flared end further comprises a first inner shoulder extending between the first portion and the first crest.

19. The transluminal stent of claim 16, wherein a diameter of the first circumferential ridge is larger than a diameter of the first portion.

20. The transluminal stent of claim 16, wherein a diameter of the second portion is larger than a diameter of the first portion but smaller than a diameter of the first circumferential ridge.
